(19) United States
(12) Patent Application Publication
(10) Pub. No.: US 2005/0274062 A1
(43) Pub. Date: Dec. 15, 2005

(54) Candle using solidified skin care agents for fuel, and providing skin care liquid as an operational end product

(76) Inventor: Sharon Yingst, Celina, OH (US)

Correspondence Address: Michael W. Starkweather;Suite 200, 9035 South 1300 East, Sandy, UT, 84094, US

(21) Appl. No.: 11/063,432

(22) Filed: Feb. 22, 2005

Related U.S. Application Data
(60) Provisional application No. 60/578,064, filed on Jun. 9, 2004.

(51) Int. Cl. F23D 3/16; A61K 35/78

(52) U.S. Cl. 44/275; 424/757

(57) ABSTRACT

A novel candle apparatus at least includes: candle housing; candle fuel deposited in the candle housing, the candle fuel at least including non-toxic solidified therapeutic skin substance; and a candle fuel liquefier; wherein the candle fuel is adapted to, upon application of the fuel liquefier, liquefy into a therapeutic substance adapted for human skin. In one refinement, the therapeutic skin substance is massage oil. In another refinement, the therapeutic skin substance is a soap agent for cleansing skin, and the like. A therapeutic skin treatment method at least includes: a) providing a candle that at least includes, candle housing, candle fuel deposited in the candle housing, the candle fuel at least including non-toxic solidified therapeutic oil, and a candle fuel liquefier; b) liquefying the candle fuel into therapeutic oil for human skin; and c) applying to the skin, therapeutic oil produced in element b).

### CROSS-REFERENCES TO RELATED APPLICATIONS

This application claims priority to U.S. Provisional Patent Application No. 60/578,064 entitled “Massage Oil Candle” and filed on Jun. 9, 2004, by Sharon Yingst.

### BACKGROUND OF THE INVENTION

1. Field of the Invention

The present invention generally relates to candles, and specifically relates to candles having therapeutic uses.

2. Description of the Related Art

Candles are often used to create an environment conducive to calm and relaxation by combining visual effects, colors and aromas. It is common for people to receive therapeutic skin treatment-either self-administered, or administered by others-with burning candles in the vicinity. Such therapeutic skin treatments may include hand and body massages with various massage oils, and bathing or showering with therapeutic body washes and soaps.

What is desirable, but unaddressed by the prior art, is providing simple candle products which directly combine the traditional attributes and benefits of candles with therapeutic skin care agents in simple articles of manufacture. For example, U.S. Pat. No. 6,730,137 discloses candles incorporating hydrogenated vegetable oil, which candles are gentle to the touch, and can to some extent, be rubbed into the hands (and on the skin).

What is not taught or suggested by the prior art are candles which directly include massage oil or other non-toxic therapeutic skin care agents as candle fuel, and in which the candle fuel is liquefied during normal candle operation to produce usable therapeutic skin care agents, such as massage oil. It is also desirable to provide candle products with the above attributes, in which the candle fuel mixture can be liquefied either by burning a conventional wick, or in a “wickless” manner with a simple heat source.

### SUMMARY OF THE INVENTION

The present invention has been developed in response to the present state of the art, and in particular, in response to the problems and needs in the art that have not yet been fully solved by currently available liner supports. Accordingly, the present invention has been developed to provide a candle apparatus at least including: candle housing; candle fuel deposited in the candle housing, the candle fuel at least including non-toxic solidified therapeutic skin substance; and a candle fuel liquefier; wherein the candle fuel is adapted to, upon application of the fuel liquefier, liquefy into a therapeutic substance adapted for human skin.

In one refinement of the present invention, the therapeutic skin substance is massage oil. In another refinement of the present invention, the therapeutic skin substance is a soap agent for cleansing skin, and the like.

The present invention has also been developed to provide a therapeutic skin treatment method at least including: a) providing a candle that at least includes, candle housing, candle fuel deposited in the candle housing, the candle fuel at least including non-toxic solidified therapeutic oil, and a candle fuel liquefier; b) liquefying the candle fuel into therapeutic oil for human skin; and c) applying to the skin, therapeutic oil produced in element b).

The present invention has been further developed to provide a solid soap article at least including: a first layer comprising soy flakes and at least one carrier oil; and a second layer comprising soy flakes, at least one carrier oil, and glycerin.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is an isometric view of a first embodiment of the present-inventive candle apparatus;

FIG. 2 is an isometric view of a second embodiment of the present-inventive candle apparatus;

FIG. 3 is an isometric view of a third embodiment of the present-inventive candle apparatus;

FIG. 4 is a top view of a fourth embodiment of the present-inventive candle apparatus;

FIG. 5 is a side view of a soap article according to the present invention; and

FIG. 6 is a side view of the soap article of FIG. 5.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment.

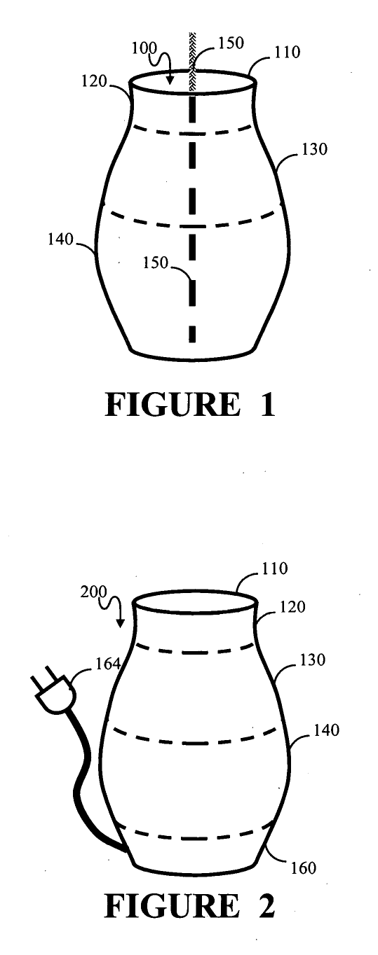

FIG. 1 illustrates a candle apparatus 100 according to a first embodiment of the present invention. A candle housing or candle container 110 contains candle fuel in the region 140. The candle housing 110 also has a region 130 for collecting liquefied massage oil (as will be described infra.), and a region 120 at the top of the housing. In the embodiment of FIG. 1, a wick 150 is used to burn and liquefy the candle fuel. The housing can be constructed of glass, ceramics, and other suitable materials, as will be appreciated by those skilled in the art.

The present invention novelly uses solidified massage oil in the candle fuel mixture, and not typical paraffin and petroleum waxes or oils. Toward that end, the candle fuel mixture contains soy oil substantially free of other vegetable oils such as cottonseed oil or hydrogenated vegetable oils. The candle fuel mixture further contains a carrier oil, which provides moisturizing and lubricating properties to liquefied oil when the candle fuel is melted. Exemplary carrier oils are jojoba oil, shea butter, and dehydrated aloe.

In operation, the candle wick 150 is ignited to burn the solid candle fuel mixture, which fuel is converted in the process to liquefied massage oil. A candle user may then use the massage oil as desired immediately after liquefication, of at a later time. The temperature of the massage oil—except immediately at the burning focal point of the wick—is designed to be tolerable to the touch.

The massage oil candle of the present invention can be constructed in a “wickless” version 200 as well. (See FIG. 2.) That is, the candle fuel liquefier takes the form of a heating source 160 rather than an ignited wick. Activation of the heating source 160 causes the candle fuel to melt and produce the aforementioned massage oil. The heating source 160 can take on many forms according to the present invention. For example, the heating source 160 can be a powered (via an electric outlet and cord 164) heating element using an ohmic heating process. Other heating sources include candle warmers and melting pots, to name a few.

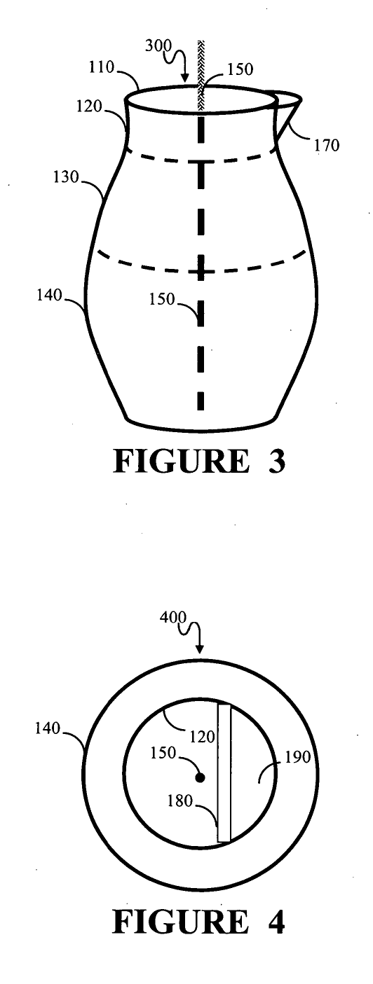

Those skilled in the art will appreciate that many variations of the candle housing are possible, given the teachings of the present invention. For example, a version 300 (see FIG. 3) of a candle apparatus contains a pouring spout 170 to facilitate pouring of the massage oil. Also, many variations of the candle housing can be constructed to provide convenient reservoirs for storing liquefied massage oil. While a simple version allows the liquefied massage oil to collect on top of the solid candle fuel mixture, other possibilities including partitioning the candle housing to allow liquefied massage oil to collect in a separate compartment from the solid candle fuel. In the version 400 of FIG. 4, a partition 180 separates the unmelted candle fuel from liquefied massage oil which collects in a reservoir 190.

An exemplary process for constructing a massage oil contains the following steps. Soy flakes are melted to about 190° F. and blended with melted shea butter having approximately one sixteenth the weight of the soy oil, and blended to uniformity. Coloring dye is added if desired. The resulting mixture is cooled to between 110° F. and 130° F., and more preferably to about 120° F., whereupon desired carrier oils (e.g., jojoba oil, dehydrated aloe mixed with heated soy oil) are added. Desired fragrance (such as for aromatherapy), other essential oils, and any other therapeutic ingredients are also added.

After the resulting mixture cools to a temperature between 95° F. and 110° F. (and more preferably about 100° F.), it is poured into a candle container/housing for hardening/solidification.

An exemplary mixture (with the range of the percent of the total weight) of ingredients for a massage oil candle according to the present invention is as follows: pure soy flakes 10-85; shea butter 0-10; dehydrated aloe and heated soy oil combined 0-20; jojoba oil 0-20; fragrance 0-5; and dye 0-1.

According to the present invention, the skin care agent used in the candle fuel mixture need not be restricted to massage oil. Rather, the skin care agent can be a soap agent. That is, as the solid candle fuel mixture melts, it is converted to a non-toxic liquefied soap which may be safely used on the human skin. While the candle fuel mixture in a “soap candle” may contain similar carrier oils found in “massage oil candles,” an additional ingredient of the soap candle fuel mixture is glycerin.

An exemplary process for manufacturing a soap candle according to the present invention uses the following steps. Soy flakes are melted and maintained between 150° F. and 190° F., and more preferably between 175° F. and 185° F. Glycerin (in and amount ranging from half the weight of soy, to two and one-half times the weight of soy) is heated to a temperature between 150° F. and 170° F., without scorching it. The soy oil and glycerin are blended to uniformity. Shea butter in an amount ranging from one sixteenth to one-eighth of the weight of soy flakes is added to the mixture. Prior to final cooling, desired carrier oils, fragrances and dyes are added to the mixture. The mixture is then cooled until it solidifies.

An exemplary mixture (with the range of the percent of the total weight) of ingredients for a soap candle according to the present invention is as follows: pure soy flakes 10-85; glycerin 25-85; jojoba oil 1-25; dehydrated aloe 1-5; shea butter 0-10; fragrance 0-5; and dye 0-1.

In the case of either the massage oil candle or the soap candle, a wick is placed in the candle fuel mixture prior to cooling if a “wicked” version is desired.

As a business option, the massage oil candle and the soap candle of the present invention may be sold in a convenient package to consumers. A consumer can used liquid soap from a melted soap candle to cleanse his or her skin, and then apply massage oil or other emollients formed from a melted massage oil candle.

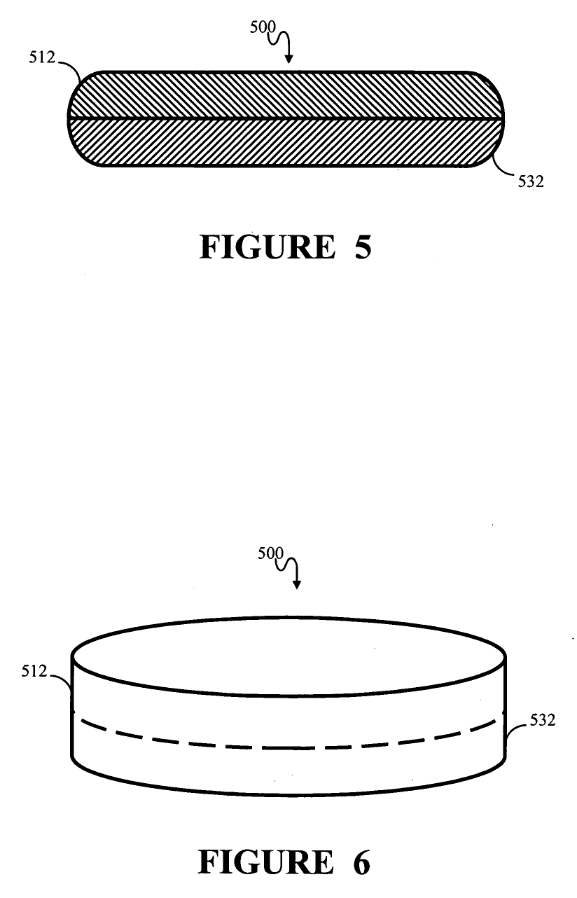

In addition to producing candle apparatuses, the present invention also applies to novel soap articles of manufacture such as the one numbered 500 in FIG. 5. According to a present-inventive manufacturing process, a soap article is produced with two distinct layers in which one layer 512 is primarily directed to cleansing and softening the skin, and another layer 532 is primarily directed to exfoliating and moisturizing the skin. An isometric view of the soap article 500 is illustrated in FIG. 6.

The layered soap article 500 of the present invention is produced according to the following exemplary steps. Soy flakes are melted at a temperature between 160° F. and 200° F. Next, glycerin and a carrier oil such as jojoba oil, shea butter, or aloe oil, and vitamin E are added to the melted soy oil. The mixture is blended to uniformity at temperatures between 140° F. and 200° F., and preferably between 140° F. and 180° F., and more preferably between 160° F. and 180° F. Colorants and fragrances are then added to the mixture, and then blended to uniformity. The resulting mixture is then poured in a mold, whereupon two separate layers form by spontaneous separation upon cooling.

An exemplary mixture (with the range of the percent of the total weight) of ingredients for an exfoliating and moisturizing soap article is as follows: soy flakes 5-30; glycerin 25-85; shea butter 0-10; jojoba oil 0-20; dehydrate aloe 0-20; vitamin E 0-2; and an exfoliating ingredient 0-1.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

It is expected that there could be numerous variations of the design of this invention.

Finally, it is envisioned that the components of the device may be constructed of a variety of materials.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

1. A candle apparatus comprising:
candle housing;
candle fuel deposited in said candle housing, said candle fuel comprising non-toxic solidified therapeutic skin substance; and
a candle fuel liquefier;
wherein said candle fuel is adapted to, upon application of said fuel liquefier, liquefy into a therapeutic substance adapted for human skin.

2. The apparatus of claim 1, wherein said therapeutic skin substance comprises massage oil.

3. The apparatus of claim 1, wherein said candle fuel liquefier comprises a candle wick operationally extended into said candle fuel, and adapted to be ignited.

4. The apparatus of claim 1, wherein said candle fuel liquefier comprises a heating source operatively coupled to said candle housing and said candle fuel.

5. The apparatus of claim 3, wherein said heating source is of the ohmic variety.

6. The apparatus of claim 1, wherein said candle housing further comprises a reservoir for collecting liquefied oil.

7. The apparatus of claim 1, wherein said candle housing comprises a spout to facilitate pouring liquefied oil.

8. The apparatus of claim 2, wherein said candle fuel comprises:
soy oil; and
at least one carrier oil.

9. The apparatus of claim 8, wherein said carrier oil comprises jojoba oil.

10. The apparatus of claim 8, wherein said carrier oil comprises shea butter.

11. The apparatus of claim 8, wherein said carrier oil comprises dehydrated aloe.

12. The apparatus of claim 1, wherein said therapeutic skin substance comprises a soap agent adapted to cleanse integumentary system tissue.

13. The apparatus of claim 12, wherein said candle fuel liquefier comprises a candle wick operationally extended into said candle fuel, and adapted to be ignited.

14. The apparatus of claim 12, wherein said candle fuel liquefier comprises a heating source operatively coupled to said candle housing and said candle fuel.

15. The apparatus of claim 12, wherein said soap agent comprises glycerin.

16. The apparatus of claim 15, wherein said soap agent further comprises soy oil.

17. The apparatus of claim 16, wherein said soap agent further comprises shea butter.

18. A therapeutic skin treatment method comprising:
a) providing a candle comprising:
candle housing;
candle fuel deposited in said candle housing, said candle fuel comprising non-toxic solidified therapeutic oil; and
a candle fuel liquefier;
b) liquefying said candle fuel into therapeutic oil for human skin; and
c) applying to the skin, therapeutic oil produced in element b).

19. The method of claim 19, wherein said therapeutic oil comprises massage oil, said method further comprising:
massaging desired areas of the body after having applied massage oil in element c).

20. A solid soap article comprising:
a first layer comprising soy flakes and at least one carrier oil; and
a second layer comprising soy flakes, at least one carrier oil, and glycerin.
